(19) United States
(12) Patent Application Publication
(10) Pub. No.: US 2009/0192440 A1
(43) Pub. Date: Jul. 30, 2009

(54) Shaped Phacoemulsification Needle Tips

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/871,863

(22) Filed: Oct. 12, 2007

Related U.S. Application Data
(60) Provisional application No. 60/829,503, filed on Oct. 13, 2006.

(51) Int. Cl. A61B 17/32; B23P 11/00

(52) U.S. Cl. 604/22; 29/428

(57) ABSTRACT

Phacoemulsification needle tips have indentations formed thereon to allow the needles to be used with torsional motion phaco handpieces and with phaco handpieces producing linear or longitudinal motion.

### PRIORITY

This application claims priority from U.S. provisional application Ser. No. 60/829,503, filed Oct. 13, 2006, which is incorporated herein by reference.

### FIELD OF THE INVENTION

This disclosure relates to surgical instruments and surgical techniques used in eye surgery and more particularly, to phacoemulsification apparatus and methods for their use.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

United States Patent Application Publication 2006/0217672 (Chon) teaches and describes a phacoemulsification tip that is swaged or crimped at its distal end. The tip is intended for use with a handpiece producing torsional motion and the reference teaches that the crimping forms cutting edges at the distal end.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

U.S. Pat. No. 6,077,285 (Boukhny) teaches and describes a torsional ultrasound handpiece configured to impart both longitudinal and torsional motion to a phacoemulsification needle.

U.S. Pat. No. 6,402,769 (Boukhny) is a continuation in part of the '285 patent and further particularizes the frequencies at which the crystals providing both the torsional and longitudinal motion are activated.

I have determined that improved results can be achieved using high-speed handpieces in both the longitudinal and torsional direction if the phacoemulsification tip is provided with a particular geometry. I have also determined that these improved results can be achieved using the straight phacoemulsification needle configuration, a configuration which is favored by a considerable number of doctors.

In accordance with these criteria, I have designed a series of tips that are specifically configured to enhance the emulsifying defect created by the handpiece in the torsional direction. In addition, the improved results are anticipated with the use of handpieces providing longitudinal motion.

In accordance with an example of the apparatus, a phacoemulsification needle is provided for use with a high-frequency torsional phacoemulsification handpiece as well as a longitudinal handpiece with the needle having a needle tip having a series of indentations formed on the tip proximate the tip opening.

In a second example, the tip is formed with straight walls and a series of indentations impressed into the walls proximate the tip opening.

In yet another example of the present invention, the tip is formed with a tapered wall portion and indentations are formed along the tapered portion of the wall proximate the tip opening.

While the following describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures

### BRIEF DESCRIPTION OF THE DRAWINGS

These and further aspects of the present invention will be best understood by reference to the accompanying drawings wherein:

FIG. 1 is a drawing showing prior art oval and square-shaped tips;

FIG. 2 is a drawing showing several prior art needle cross-sectional configurations;

FIG. 3 is a partial lateral sectional view of a phacoemulsification needle tip with the tip walls being unindented proximate the tip opening;

FIG. 4 is a view along line 4-4 of FIG. 3;

FIG. 5 is a schematic view of the tip of FIG. 3 showing the tip positioned between two forming die blocks;

FIG. 6 is a view along 6-6 of FIG. 5 also showing the bend parameters to be met by the forming die blocks;

FIG. 7 is a partial schematic view showing the forming die blocks contacting the tip of FIG. 3;

FIG. 8 is a schematic view showing the operation of the forming die blocks as viewed along line 8-8 of FIG. 7;

FIG. 9 is a schematic view showing the tip of FIG. 7 rotated 90 degrees and the forming die blocks positioned to perform a second operation;

FIG. 10 is a view along line 10-10 of FIG. 9;

FIG. 11 is a schematic view showing the forming die blocks forming another set of indentations in the tip of FIG. 3;

FIG. 12 is a view along line 12-12 of FIG. 11;

FIG. 13 is a partial lateral sectional view showing the completed tip;

FIG. 14 is a view along line 14-14 of FIG. 13;

FIG. 15 is a schematic view showing the tip of FIG. 3 positioned between angled forming die blocks;

FIG. 16 is a partial sectional view showing the tip of FIG. 15 with opposed indentations formed proximate the tip opening;

FIG. 17 is a top view showing the formation of four indentations

FIG. 18 is a partial sectional view of the tip of FIG. 17;

FIG. 19 is a top schematic view of an unindented tip;

FIG. 20 is a schematic sectional view of the tip of FIG. 19 showing the placement of a forming block;

FIG. 21 is a top schematic view of FIG. 20 showing the positioning of four forming die blocks;

FIG. 22 is a partial schematic cross-sectional view showing the positioning of two forming die blocks;

FIG. 23 is a top schematic view showing the formation of indentations by four forming die blocks;

FIG. 24 is a partial schematic cross-sectional view of FIG. 23;

FIG. 25 is a top schematic view of FIG. 23 with the forming die blocks removed; and

FIG. 26 is a partial schematic sectional view along 26-26 of FIG. 25.

### DETAILED DESCRIPTION OF THE INVENTION

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

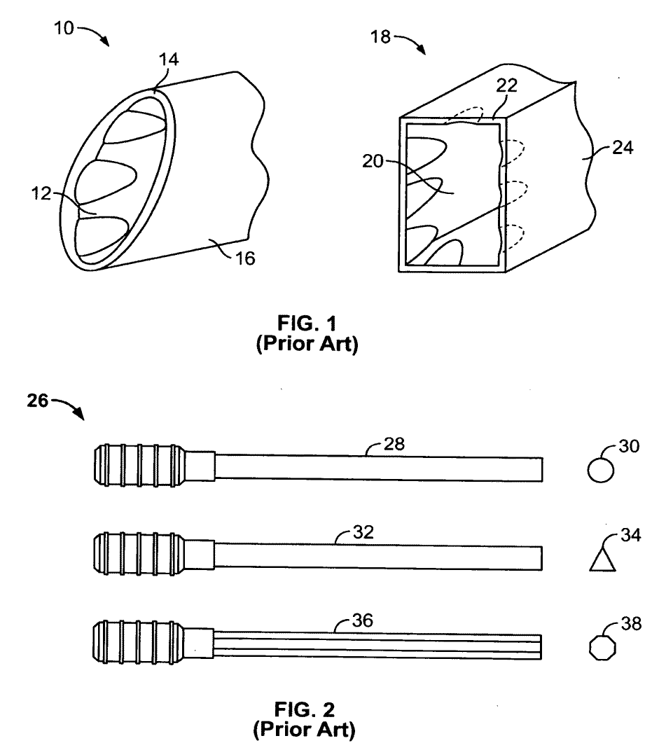

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38.

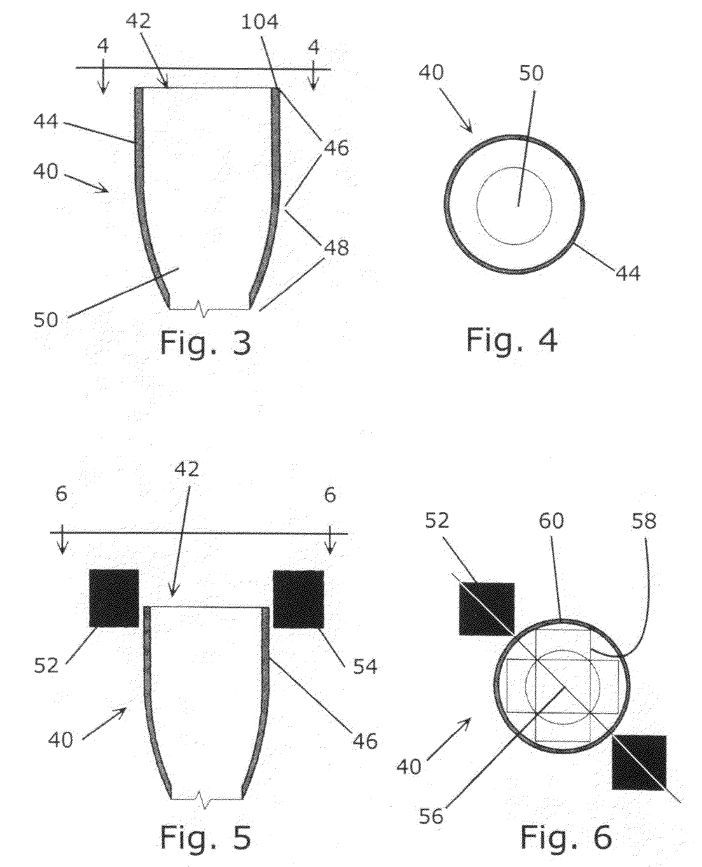

Referring now to FIG. 3, the numeral 40 indicates generally a phacoemulsification needle embodying certain aspects of the present invention.

In FIG. 3, the numeral 40 identifies generally a partial sectional view of a phacoemulsification tip having a tip opening 42 defined by a lip 104 and having a side wall 44 along which a first portion 46 thereof is formed with straight wall, that is, with a cross-sectional configuration of portion 46 as a right circular section, and a second wall portion 48 tapered to form a throat 50. Throat 50 is also depicted in FIG. 4.

Referring now to FIG. 5, a pair of forming die blocks 52, 54 are placed with tip 40 positioned such that a portion of straight portion 46, proximate to opening 42, is positioned between die blocks 52, 54.

Referring to FIG. 6, a view along 6-6 of FIG. 5 is shown wherein die blocks 52, 54 are positioned along an extended diameter 56 of tip 40. FIG. 6 also has a grid pattern 58 superimposed thereon showing the ultimate configuration of the bent portion 60 of tip 40. Bent portion 60 is understood to be that portion of straight portion 46 that is contacted by die blocks 52, 54.

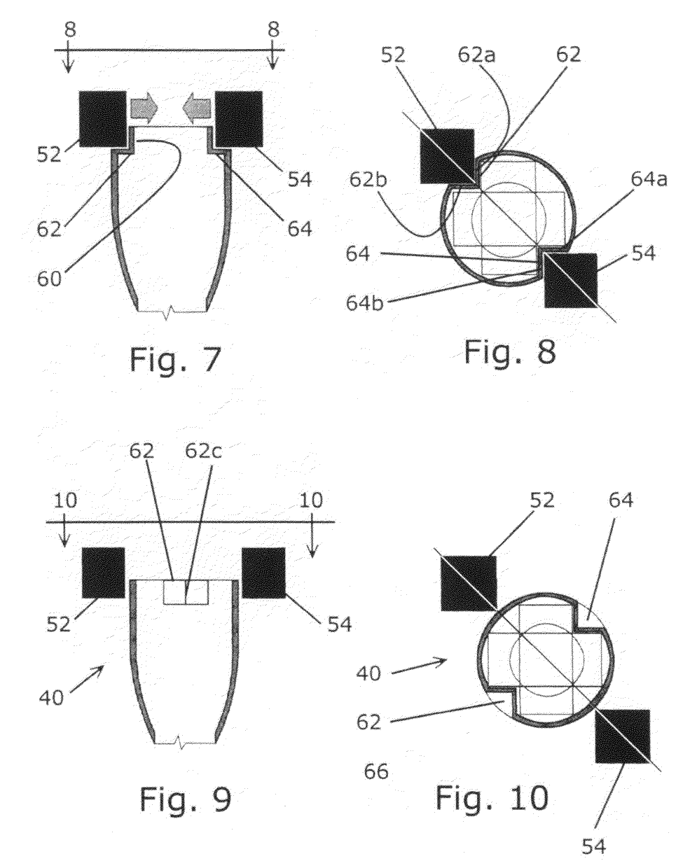

Referring now to FIG. 7, die blocks 52, 54 are shown as having been moved toward one another to contact tip 40 at portion 60 to form a first indentation 62 and a second indentation 64. Referring to FIG. 8, the position of die blocks 52, 54 after the first forming step is depicted with indentations 62, 64 shown as generally V-shaped and having substantially planar sides 62a, 62b and 64a, 64b respectively. It is anticipated that the angle between sides 62a and 62b and between sides 64a and 64b would be approximately 90° although other angles can also be selected.

Referring now to FIG. 9, tip 40 is shown as rotated 90 degrees between die blocks 52, 54 with first indentation 62 shown and with die blocks 52, 54 positioned to complete the formation of a second pair of indentations.

Referring now to FIG. 10, die blocks 52, 54 are shown positioned along a second extended diameter 66 which, in this example, is at a 90° angle to first extended diameter 56. Previously formed indentations 62, 64 are also shown.

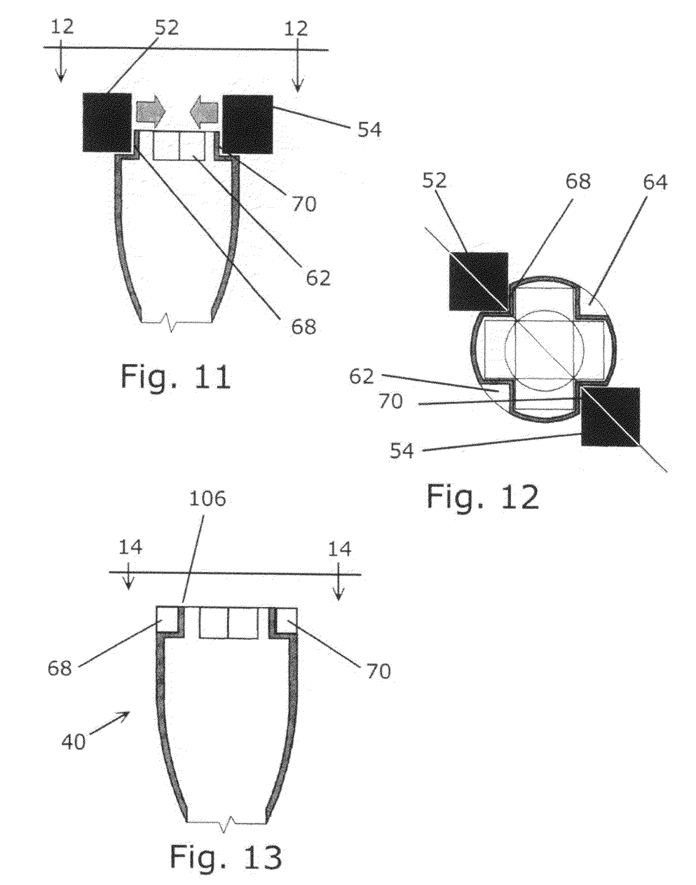

Referring now to FIG. 11, die blocks 52, 54 are shown positioned after the formation of a second set of indentations with the positioning of die blocks 52, 54 also shown in FIG. 12.

Referring now to FIG. 13, a partial lateral sectional view of tip 40 is shown with the manufacturing or indentation process completed and with indentations 68 and 70 shown.

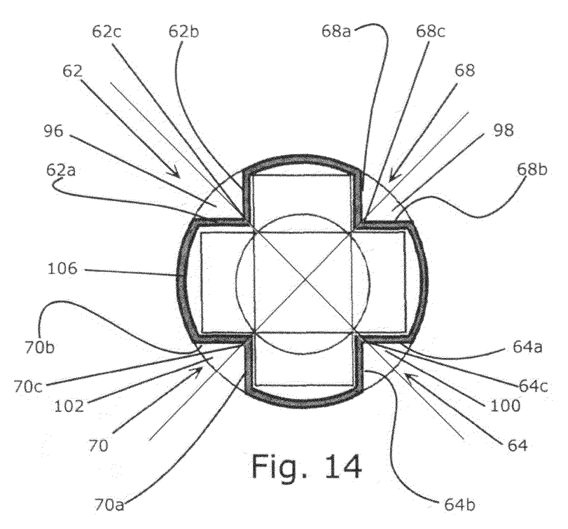

In FIG. 14, a top view is shown with the upper wall portion 60 with finished indentations 62, 68, 64 and 70 shown. In this view it is seen that indentation 62 has sides 62a and 62b meeting at a crease 62c, indentation 64 has sides 64a and 64 b meeting at a crease 64c, indentation 68 has sides 68a and 68b meeting at a crease 68c, and indentation 70 has sides 70a and 70b meeting at a crease 70c. Indentation 62 also has a land or “floor” 96 extending between sides 62a and 62b, indentation 64 has a land 100 extending between sides 64a and 64c, indentation 68 has a land 98 extending between sides 68a and 68c, and indentation 70 has a land 102 extending between sides 70a and 70c.

Consistent with the foregoing description, in this example, preferably the angle between sides 68a and 68b and 70a and 70b is approximately 90°.

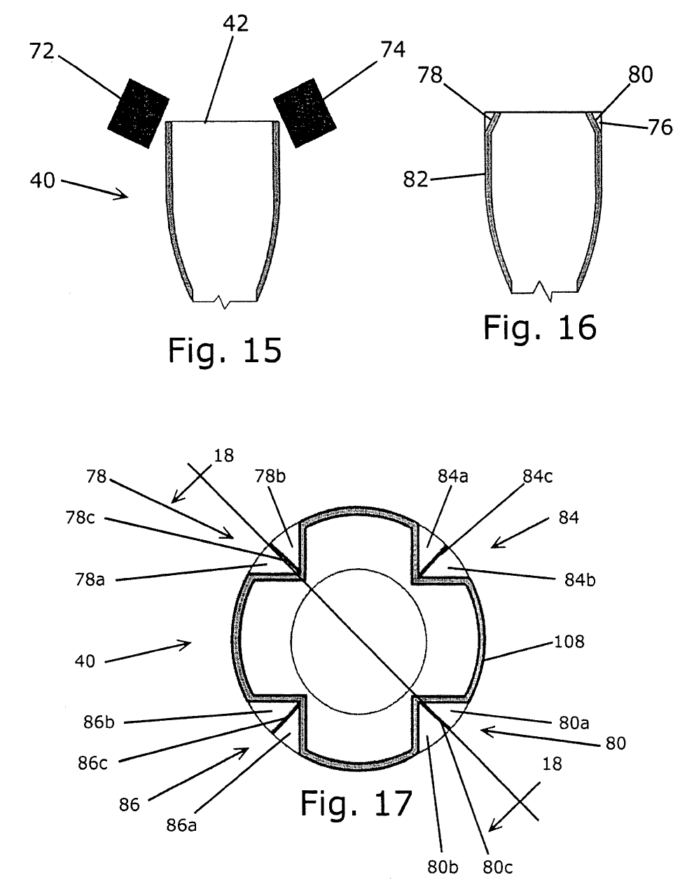

Referring now to FIG. 15, tip 40 is shown in the same configuration shown in FIG. 3 with a pair of angled die blocks 72, 74 positioned proximate tip opening 42. In FIG. 16, when die blocks 72, 74 are advanced to contact upper portion 76 of tip 40, angled indentations 78, 80 are formed in side wall 82 at the upper periphery of tip 40 as defined by section 76.

Tip 40 is then rotated and positioned between die blocks 78, 80 which are oriented to create a second set of indentations 84, 86 (shown in FIG. 17) in the same fashion as described in connection with FIGS. 3-14.

Referring now to FIG. 17, a top view of the completed tip of FIG. 15 is shown with indentations 78, 80, 84, and 86, it being understood that each indentation pair 78, 80 and 84, 86 are formed in the same manner as described hereinabove, are formed in pairs, and, in this example, are formed with an included angle of approximately 90°. As described above, indentation 78 has walls 78a and 78b meeting at crease 78c, indentation 80 has walls 80a and 80b meeting at crease 80c, indentation 84 has walls 84a and 84b meeting at crease 84c, and indentation 86 has walls 86a and 86b meeting at crease 86c. Preferably, creases 78c, 80c, 84c and 86c are formed inward at an angle less than 90° to wall section 76.

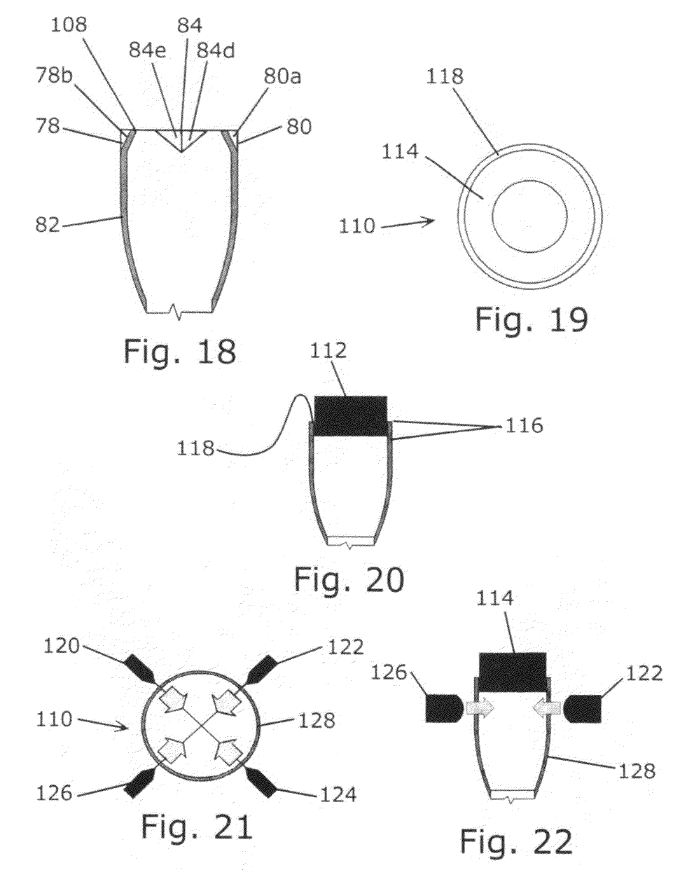

Referring now to FIG. 18, a partial lateral sectional view of tip 40 is shown with indentation 84 seen from the inside of tip 40 with the rear 84d of side 84a and the rear 84e of side 84b.

Referring to FIGS. 13 and 18 the numerals 106, 108 identify, respectively, lip 104 after the associated manufacturing processes have been completed. Lips 106, 108 may if desired be polished smooth to reduce the possibility that contact between lip 106, 108 will cause damage to any eye tissue

Referring now to FIG. 14, sides 62a, 62b, 64a, 64b, 68a, 68b, 70a and 70b, together with creases 62c, 64c, 68c and 70c form cutting or emulsifying surfaces of tip 40 when tip 40 is used in a hand piece that moves tip 40 in a torsional direction.

Referring now to FIG. 14, lip 106, as it is included in indentations 62, 64, 68 and 70, together with lands 96, 98, 100 and 102 form cutting or emulsifying surfaces when used in a handpiece imparting longitudinal motion to tip 40.

Referring now to FIG. 17, walls 78a, 78b, 80a, 80b, 84a, 84b, 86a and 86b form cutting or emulsifying surfaces when used in a handpiece imparting torsional motion to tip 40.

Referring now to FIG. 17, lip 108 as it is included in indentations 78, 80, 84 and 86 forms a cutting or emulsifying surface when tip 40 is used in a handpiece that imparts longitudinal motion to tip 40.

Referring now to FIGS. 19 through 26 another example of a shaped phaco tip is described wherein the indentations are formed spaced downwardly from the lip.

As seen in FIGS. 19 and 20 a tip 110 has a mouth 114 with a lip 118. A block 112 is inserted into mouth 114. As seen in FIG. 20 block 112 extends into mouth 114 a distance 116 past lip 118.

Referring now to FIGS. 21 and 22, forming die blocks 120, 122, 124 and 126 are positioned about the outer wall 128 of tip 110. The example shown has four such die blocks spaced 90° apart to from four indentations but it should be understood that other numbers and configurations of indentations may be selected as well. In FIG. 22 it can be seen that die blocks 122, 126 are positioned to contact wall 128 at points below the distance 116 to which block 112 is inserted into tip 110. In this example, die blocks 120, 122, 124 and 126 are positioned to contact wall 128 at the same distance 116 down from lip 118.

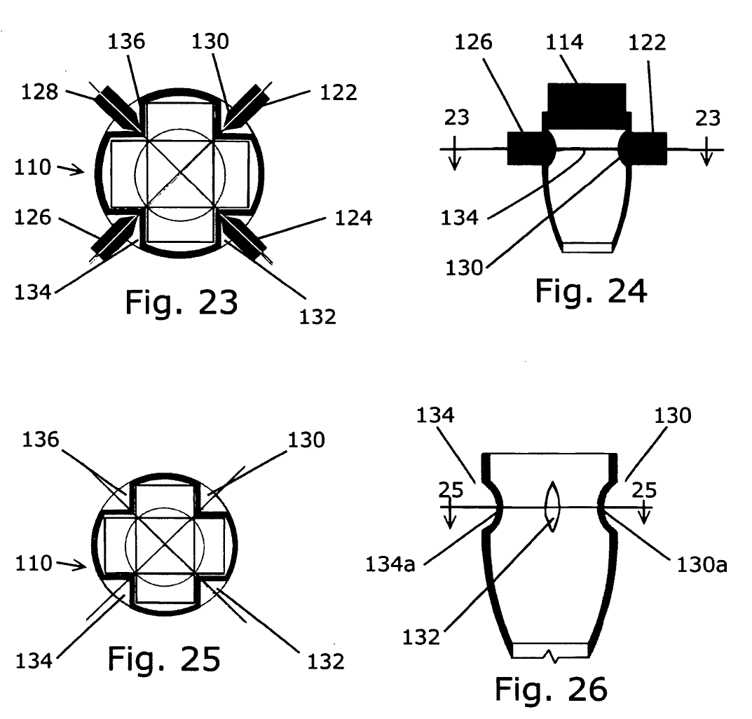

Referring now to FIGS. 23 and 24 the forming process is illustrated. FIG. 23 is a view along 23-23 of FIG. 24. As die blocks 120, 122, 124 and 126 are moved axially inwardly they deform wall 128 to form, respectively, indentations 130, 132, 134 and 136. FIG. 24 illustrates the formation and configuration of indentations 130 and 134 by die blocks 122, 126.

Referring now to FIGS. 25 and 26 the completed tip 110 is shown with block 114 removed. FIG. 25 is a view along 25-25 of FIG. 26. In this example, indentations 130, 132, 134 and 136 are somewhat elliptical in shape and form a somewhat curvilinear surface. As an example, surface 130a of indentation 130 and 134a of indentation 134 form surfaces utilized in torsional motion to increase the efficiency of phacoemulsification, while lip 118 is usable with handpieces employing longitudinal motion.

I claim:

1. A phacoemulsification needle, said needle of the type made to be mounted on a phacoemulsification handpiece, said needle comprising:
a hollow needle shaft adapted at a proximal end to be attached to said handpiece;
a hollow needle tip formed at a distal end of said shaft
said tip having a wall terminating in a lip,
said lip defining a mouth;
means formed on said tip to form at least one cutting or emulsifying surface when said handpiece imparts longitudinal motion to said needle; and
means formed on said tip to form at least one cutting or emulsifying surface when said handpiece imparts torsional motion to said needle.

2. The apparatus as recited in claim 1 wherein at least one said torsional cutting surface is formed at said lip.

3. The apparatus as recited in claim 2 wherein said torsional cutting surface comprises an indentation formed in said tip wall and including a portion of said lip.

4. The apparatus as recited in claim 3 wherein said indentation has a pair of planar sides meeting at a crease.

5. The apparatus as recited in claim 4 wherein said planar sides form an angle of about 90° with said crease.

6. The apparatus as recited in claim 4 wherein said planar sides meet said lip at an angle of about 90°.

7. The apparatus as recited in claim 4 wherein said crease is formed to be approximately parallel to said tip wall.

8. The apparatus as recited in claim 4 wherein said crease is formed at an angle less than 90° to said tip wall.

9. The apparatus as recited in claim 3 wherein four said indentations are formed spaced about said tip wall.

10. The apparatus as recited in claim 4 wherein said indentation further comprises a land formed between said planar sides distal from said lip.

11. The apparatus as recited in claim 10 wherein said longitudinal cutting means comprises said lands and said lip.

12. The apparatus as recited in claim 1 wherein at least one said torsional cutting surface is formed distal from said lip.

13. The apparatus as recited in claim 12 wherein said torsional cutting surface comprises an indentation formed in said tip wall.

14. The apparatus as recited in claim 12 wherein said longitudinal cutting surface comprises said lip.

15. A method for manufacturing a phacoemulsification needle for use with phacoemulsification handpieces that impart a torsional or longitudinal motion to said needle, said needle of the type including a hollow needle shaft adapted at a proximal end to be attached to said handpiece, a hollow needle tip formed at a distal end of said shaft, said tip having a wall terminating in a lip, said lip defining a mouth, said method comprising the steps of:
positioning said needle tip proximate at least one die block; and
forming at least one indentation on said wall by advancing said die block to contact said wall at said lip.

16. A method for manufacturing a phacoemulsification needle for use with phacoemulsification handpieces that impart a torsional or longitudinal motion to said needle, said needle of the type including a hollow needle shaft adapted at a proximal end to be attached to said handpiece, a hollow needle tip formed at a distal end of said shaft, said tip having a wall terminating in a lip, said lip defining a mouth, said method comprising the steps of:
positioning said needle tip proximate two opposed of said die blocks; and
forming a first pair of opposed said indentations by advancing said die blocks to contact said wall at said lip.

17. The method of claim 16 including the step of:
forming said indentations to include a pair of substantially planar sides meeting at a crease.

18. The method of claim 16 including the steps of:
rotating said tip 90° between said blocks; and
advancing said blocks to form a second pair of said indentations.

19. A method for manufacturing a phacoemulsification needle for use with phacoemulsification handpieces that impart a torsional or longitudinal motion to said needle, said needle of the type including a hollow needle shaft adapted at a proximal end to be attached to said handpiece, a hollow needle tip formed at a distal end of said shaft, said tip having a wall terminating in a lip, said lip defining a mouth, said method comprising the steps of:
positioning said needle tip proximate at least one die block; and
forming at least one indentation on said wall by advancing said die block to contact said wall at a point distal from said lip.

20. The method of claim 19 including the steps of:
inserting a block a selected distance into said mouth prior to advancing said blocks; and
forming at least one said indentation by advancing said die blocks to contact said wall at a point greater than said selected distance distal from said lip.

21. A method for manufacturing a phacoemulsification needle for use with phacoemulsification handpieces that impart a torsional or longitudinal motion to said needle, said needle of the type including a hollow needle shaft adapted at a proximal end to be attached to said handpiece, a hollow needle tip formed at a distal end of said shaft, said tip having a wall terminating in a lip, said lip defining a mouth, said method comprising the steps of:
inserting a block a selected distance into said mouth prior to advancing said blocks;
positioning said needle tip proximate two opposed pairs of said die blocks; and
forming first and second pairs of opposed said indentations by advancing said die blocks to contact said wall at a point greater than said distance distal from said lip.
